(19) United States
(12) Patent Application Publication
(10) Pub. No.: US 2022/0080067 A1
(43) Pub. Date: Mar. 17, 2022

(54) Bag Disinfection

(71) Applicant: Ali Aldahaim, Al Jahra (KW)

(72) Inventor: Ali Aldahaim, Al Jahra (KW)

(21) Appl. No.: 17/527,820

(22) Filed: Nov. 16, 2021

Related U.S. Application Data
(63) Continuation-in-part of application No. 17/411,162, filed on Aug. 25, 2021.
Application No. 17/411,162 is a continuation-in-part of application No. PCT/IB2020/058639, filed on Sep. 16, 2020.

(51) Int. Cl. A61L 2/18; A61L 2/26; B65D 65/46; B65D 65/40; B65D 33/16

(52) U.S. Cl. CPC A61L 2/186; A61L 2/26; B65D 65/466; A61L 2202/122; B65D 33/16; A61L 2202/16; B65D 65/40

(57) ABSTRACT

A bag for sterilizing and disinfecting personal items is provided, comprisinga layer comprising a disinfectant wet wipe, alone or in combination with another layer comprising an environmentally friendly degradable plastic linoleum, particularly wherein the wet wipe comprises cotton or tree fibers and is wetted with: i) one or more disinfectants approved by the World Health Organization; or ii) water and one or more extracts from fruits and/or herbs; wherein the bag is configured to enable a user to rub one or more personal items placed inside the bag from outside the bag, thereby sterilizing and disinfecting the one or more personal items without the need for the user to touch the one or more personal items. Methods for sterilizing and disinfecting a personal item are also provided.

### CROSS-REFERENCE TO RELATED APPLICATIONS

The present invention is a U.S. Continuation-in-Part Patent Application that claims priority to U.S. Continuation-in-Part patent application Ser. No. 17/411,162, entitled “Bag Disinfection,” filed on Aug. 25, 2021; which claims priority to International Patent Application No. PCT/1B2020/058639, entitled “Bag Disinfection,” filed on Sep. 16, 2020; the entire disclosures of which are incorporated herein by reference.

### FIELD OF THE INVENTION

The present invention relates generally to disinfectant products and more specifically to bags for disinfecting personal items.

### BACKGROUND

Microorganisms such as bacteria, fungi, and viruses are ever-present in the environment. Microorganisms that are capable of causing infectious diseases are known as pathogens and include influenza (flu) and coronavirus disease 2019 (COVID-19), Pathogens can spread not only through contact between people but also through contact with touched surfaces. Surfaces can become contaminated either by airborne organisms landing on a surface or through direct contact via touch.

Frequently touched objects such as personal items cell phones, keys, and the like), come into direct contact with the users' hands and become a source of contamination of potentially pathogenic microorganisms. Users may acquire pathogens following direct contact with the contaminated personal belonging followed by subsequent hand-to-mouth, hand-to-nose, and/or hand-to-eye contact. The COVID-19 pandemic has highlighted widespread shortcomings with respect to the public's adherence to public health recommendations, which adds an urgency to the development of new and innovative ideas for preventing the spread of pathogens and reducing infections.

Therefore, a need exists for improved products and methods to prevent the spread of pathogens and reduce infections, particularly for improved products and methods relating to disinfecting personal items.

### SUMMARY

To address the foregoing problems, in whole or in part, and/or other problems that may have been observed by persons skilled in the art, the present disclosure provides compositions and methods as described by way of example as set forth below.

A bag for sterilizing and disinfecting personal items is provided, comprising:

a) a first layer comprising an environmentally friendly degradable plastic linoleum;

b) a second layer comprising flexible glass fiber insulation or any suitable insulator, wherein the insulation or suitable insulator provides insulation and support for the first layer and is preferably environmentally friendly and biodegradable; and

c) a third layer comprising a disinfectant wet wipe wetted with:
i) one or more disinfectants approved by the World Health Organization; or
ii) water and one or more extracts from fruits and/or herbs;
   wherein the bag is configured to enable a user to rub one or more personal items placed inside the bag from outside the bag, thereby sterilizing and disinfecting the one or more personal items without the need for the user to touch the one or more personal items. In some embodiments, the one or more disinfectants approved by the World Health Organization comprise chlorine-based products, ethanol, and/or hydrogen peroxide. In other embodiments, the wet wipe comprises cotton or tree fibers. In other embodiments, the bag comprises a lock.

In one embodiment, a bag for sterilizing and disinfecting personal items is provided comprising:

a) a first layer comprising an environmentally friendly degradable plastic linoleum; andb) a second layer comprising a disinfectant wet wipe wetted with:
i) one or more disinfectants approved by the World Health Organization; or
   ii) water and one or more extracts from fruits and/or herbs;wherein the bag is configured to enable a user to rub one or more personal items placed inside the bag from outside the bag, thereby sterilizing and disinfecting the one or more personal items without the need for the user to touch the one or more personal items.

In another embodiment, the bag for sterilizing and disinfecting personal items comprises a layer comprising a disinfectant wet wipe reinforced with cotton or tree fibers and wetted with:
i) one or more disinfectants approved by the World Health Organization; or
   ii) water and one or more extracts from fruits and/or herbs;wherein the bag is configured to enable a user to rub one or more personal items placed inside the bag from outside the bag, thereby sterilizing and disinfecting the one or more personal items without the need for the user to touch the one or more personal items.

In one embodiment, the personal items comprise a mobile device, wherein the bag is sized to accommodate a desired make and model of mobile device.

In another embodiment, the personal items comprise various articles and the bag is a shopping bag sized to accommodate and the various articles' shapes and sizes. The bag may further comprise a handle configured to enable easy loading on the palm of the user's hand and is sized according to a desired number and size of personal items to be sterilized and disinfected while being transported.

In another embodiment, the personal items comprise bottled beverages or canned beverages, wherein the hag is sized to accommodate the bottled beverages' or canned beverages' shapes and sizes.

In another embodiment, the bag is divided into a first section comprising a wet wipe wetted with disinfecting materials approved by the World. Health Organization, and a second section comprising a wet wipe wetted with water and materials extracted from herbs and/or fruits, thereby enabling a user to rinse or purify personal items that have been treated in the first section.

In another embodiment, the bag is divided into multiple sections of varying size, wherein each section comprises the first layer, the second layer, and the third layer. Some of the multiple sections may comprise disinfectant wet wipes wetted with one or more disinfectants approved by the World Health Organization, and some of the multiple sections may comprise disinfectant wet wipes wetted with water and one or more extracts from fruits and/or herbs.

A method for sterilizing and disinfecting a personal item is also provided, comprising placing the personal item in the bag of any of the bags described herein and rubbing the bag with one or both hands; thereby sterilizing and disinfecting the personal item without the need for the user to touch the one or more personal items. In some embodiments the personal item is a mobile device and the bag is sized to receive the mobile device. In another embodiment, the personal item comprises various articles and the bag is a shopping bag sized to receive the various articles. In another embodiment, the personal item comprises a bottled beverage or a canned beverage and the bag is sized to receive the various articles the bottled beverage or the canned beverage. In another embodiment, the bag comprises multiple sections of varying size, wherein some of the multiple sections comprise disinfectant wet wipes wetted with one or more disinfectants approved by the World Health Organization and wherein some of the multiple sections comprise disinfectant wet wipes wetted with water and one or more extracts from fruits and/or herbs.

Additional features of the invention will be or will become apparent to one with skill in the art upon examination of the following figures and detailed description. It is intended that all such additional features and advantages be included within this description, be within the scope of the invention, and be protected by the accompanying claims.

### BRIEF DESCRIPTION OF THE DRAWINGS

Having thus described the subject matter of the present invention in general to reference will now be made to the accompanying drawings, which are not necessarily drawn to scale.

FIG. 1 shows a bag for sterilizing and disinfecting personal items in accordance with an embodiment of the present invention. FIG. 1A shows a side view of the bag; FIG. TB shows a front view of the bag; and FIG. 1C shows a front see-through view of the bag.

FIG. 2 shows a bag for sterilizing and disinfecting personal items in accordance with another embodiment of the present invention. FIG. 2A shows a side view of the bag;

FIG. 2B shows a front view of the bag; and FIG. 2C shows a front see-through view of the bag.

FIG. 3 shows a bag for sterilizing and disinfecting personal items in accordance with another embodiment of the present invention. FIG. 3A shows a side view of the bag;

FIG. 3B shows a front view of the bag; FIG. 3C shows a front see-through view of the bag into which a bottled beverage has been inserted; and FIG. 31) shows a front see-through view of the bag into which a canned beverage has been inserted.

FIG. 4 shows a bag for sterilizing and disinfecting personal items in accordance with another embodiment of the present invention in which the bag comprises two separate pockets.

FIG. 5 shows a bag for sterilizing and disinfecting personal items in accordance with another embodiment of the present invention in which the bag comprises multiple pockets of varying size.

### DETAILED DESCRIPTION

The subject matter of the present invention now will be described more fully hereinafter with reference to the accompanying drawings, in which some, but not all embodiments of the subject matter of the present invention are shown. Like numbers refer to like elements throughout. The subject matter of the present invention may be embodied in many different forms and should not be construed as limited to the embodiments set forth herein; rather, these embodiments are provided so that this disclosure will satisfy applicable legal requirements. Indeed, many modifications and other embodiments of the subject matter of the present invention set forth herein will come to mind to one skilled in the art to which the subject matter of the present invention pertains having the benefit of the teachings presented in the foregoing descriptions and the associated drawings. All illustrations of the drawings are for the purpose of describing selected versions of the present invention and are not intended to limit the scope of the present invention. Therefore, it is to be understood that the subject matter of the present invention is not to be limited to the specific embodiments disclosed and that modifications and other embodiments are intended to be included within the scope of the appended claims.

### Bag Disinfection

The present invention is a bag for sterilizing and disinfecting personal items such as mobile devices, sunglasses, eyeglasses, wallets, beverage containers, and the like. The bag is designed for easy use, even by children, the elderly, and the partially disabled. The bag is also designed for sterilizing and disinfecting personal items in the best, fastest, and safest way possible without the need for additional devices.

The bag of the present invention is designed in different sizes that vary according to the types of personal items to be sterilized and disinfected. The bag comprises layers from the outside to the inside comprising:

1) a first layer comprising an environmentally friendly degradable plastic linoleum;

2) a second layer comprising flexible glass fiber insulation or any suitable insulator, wherein the insulation or suitable insulator provides insulation and support for the first layer and is preferably environmentally friendlyand biodegradable; and

3) a third layer comprising a disinfectant wet wipe wetted with one or more disinfectants approved by the World Health Organization;

wherein the bag is configured to enable a user to rub one or more personal items placed inside the bag from outside the bag, thereby sterilizing and disinfecting the one or more personal items without the need for the user to touch the one or more personal items.

The selection of disinfectants should take account of any particular microorganisms to be targeted, as well as the recommended concentration and contact time, the compatibility of the chemical disinfectants and surfaces to be tackled, toxicity, ease of use, and stability of the product. Exemplary disinfectants include, but are not limited to, chlorine-based products (e.g., hypochlorite) at about 0.1% (1000 ppm) for general environmental disinfection), ethanol at about 70%-90%, or hydrogen peroxide ≥0.5%. Contact time of a minimum of 1 minute is recommended for these disinfectants (Risk Communication and Community Engagement (RCCE) Action Plan Guidance COVID-19 Preparedness and Response, Geneva: World Health Organization (2020); Cleaning and Disinfection of Environmental Surfaces in the Context of COVID-19, Interim Guidance, Geneva: World Health Organization (May 15, 2020)).

Alternatively, in some embodiments where light cleansing of the personal items is desired, the disinfectant wet wipe is wetted with water and one or more extracts from fruits and/or herbs.

In some embodiments, the wet wipe comprises cotton or tree fibers.

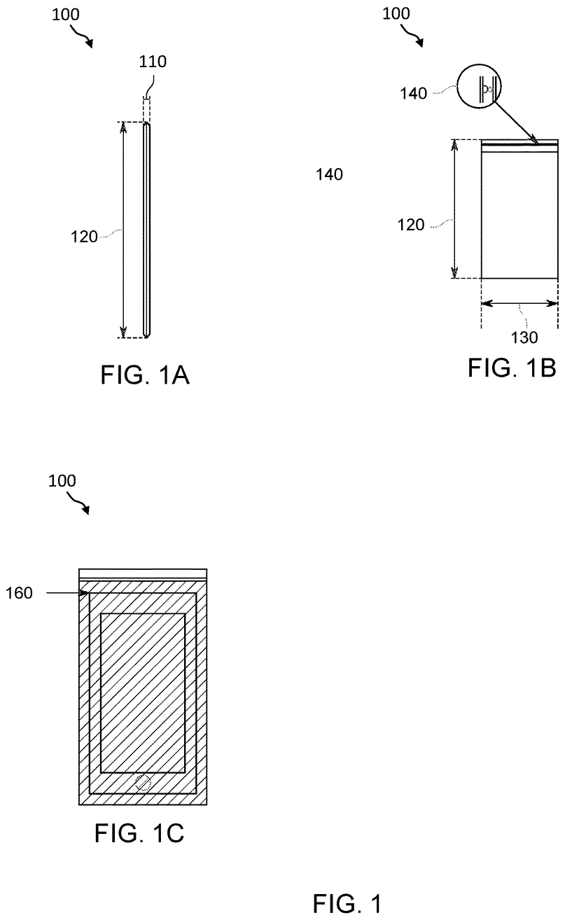

Referring now to FIG. 1, a bag 100 for sterilizing and disinfecting personal items in accordance with an embodiment of the present invention is shown, particularly for sterilizing and disinfecting a mobile device. FIG. 1A shows a side view of bag 100 having a width 110 and a height 120. Width 110 may vary according to need. Height 120 may also vary according to need such as the size of the make and model of mobile device, and particularly may be about 10 cm, about 11 cm, about 12 cm, about 13 cm, about 14 cm, about 15 cm or more.

Bag 100 may include two parts assembled in such a way that the bag is closed on three sides and open on one side and comprises a first layer, a second layer, and a third layer, wherein the first layer comprises an environmentally friendly degradable plastic linoleum; the second layer comprises flexible glass fiber insulation or any suitable insulator to provide insulation and support for the first layer; and the third layer comprises a disinfectant wet wipe wetted with one or more disinfectants approved by the World Health Organization, such as chlorine-based products, ethanol, or hydrogen peroxide. Alternatively, where light cleansing of the mobile device is desired, the disinfectant wet wipe is wetted with water and one or more extracts from fruits and/or herbs.

FIG. 1B shows a front view of bag 100 having a length 130 and height 120. Length 130 may vary according to need, and particularly may be about 4 cm, about 5 cm, about 6 cm, about 7 cm, about 8 cm, or more. The side of bag 100 that is open comprises an interlock design 140 for closing and opening the bag in a simplified way while maintaining a tight closure, thereby enabling effective sterilization and disinfection of personal items inserted into bag 100.

FIG. 1C shows a front see-through view of bag 100, into which mobile device 160 has been inserted. A user need only move or rub the bag with one or both hands until mobile device 160 is sterilized and disinfected easily and quickly.

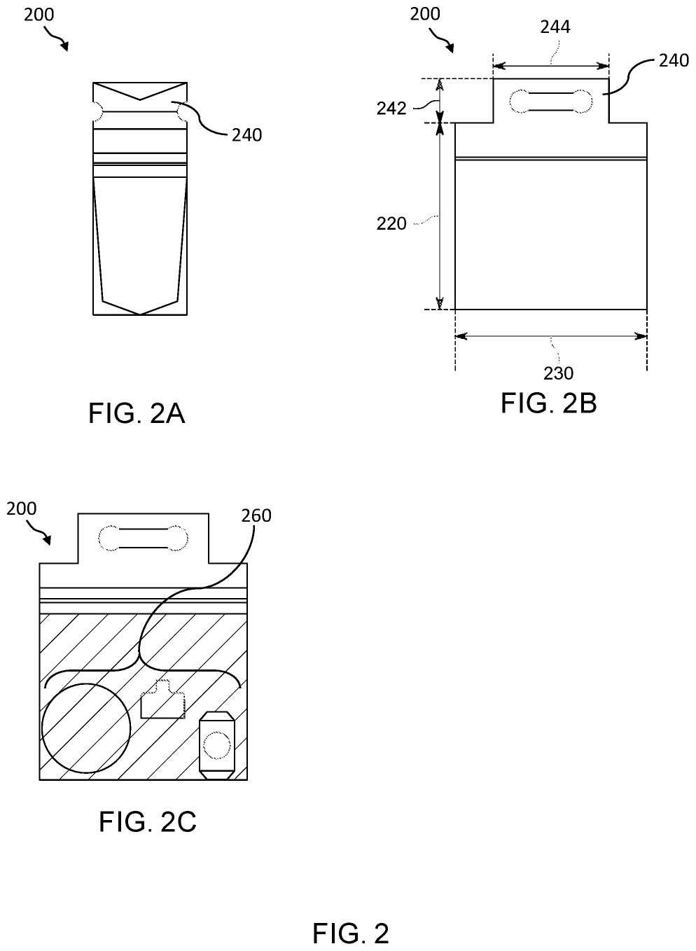

Referring now to FIG. 2, a bag 200 for sterilizing and disinfecting personal items in accordance with an embodiment of the present invention is shown, particularly for a shopping bag for sterilizing and disinfecting personal items. Shopping bags are one of the most important uses of the present invention because they are a requirement of daily life, yet many fear that shopping bags are a source of disease transmission.

FIG. 2A shows a side view of bag 200 having a handle 240. FIG. 2B shows a front view of bag 200 having a length 220 and a height 230. Length 220 and height 230 may vary according to need such as the number and size of personal items to be sterilized and disinfected while being transported, e.g. wherein bag 200 is a shopping bag for carrying various articles from a store to a car and/or to home. Alternatively, where light cleansing of the various articles is desired, the disinfectant wet wipe is wetted with water and one or more extracts from fruits and/or herbs. Light cleansing is appropriate for personal articles such as mobile phones, wallets, glasses, and the like that may be damaged by strong alcohol or other strong cleaning products.

Bag 200 may include two parts assembled in such a way that the bag is closed on three sides and open on one side and comprises a first layer, a second layer, and a third layer, wherein the first layer comprises an environmentally friendly degradable plastic linoleum; the second layer comprises flexible glass fiber insulation or any suitable insulator to provide insulation and support for the first layer; and the third layer comprises a disinfectant wet wipe wetted with one or more disinfectants approved by the World Health Organization, such as chlorine-based products, ethanol, or hydrogen peroxide.

Handle 240 has a height 242 and a length 244. Handle 240 is used to carry bag 200 and is configured to enable easy loading on the palm of the user's hand. Length 244 is proportional to height 242, which vary according to need such as the number and size of personal items to be sterilized and disinfected while being transported. Various articles 260

FIG. 2C shows a front see-through view of bag 200, into which various articles 260 have been inserted. A user only needs to nib the bag from the outside with one hand or with both hands until various articles 260 are sterilized and disinfected, and the more and finer the rubbing, the better the sterilization and disinfection.

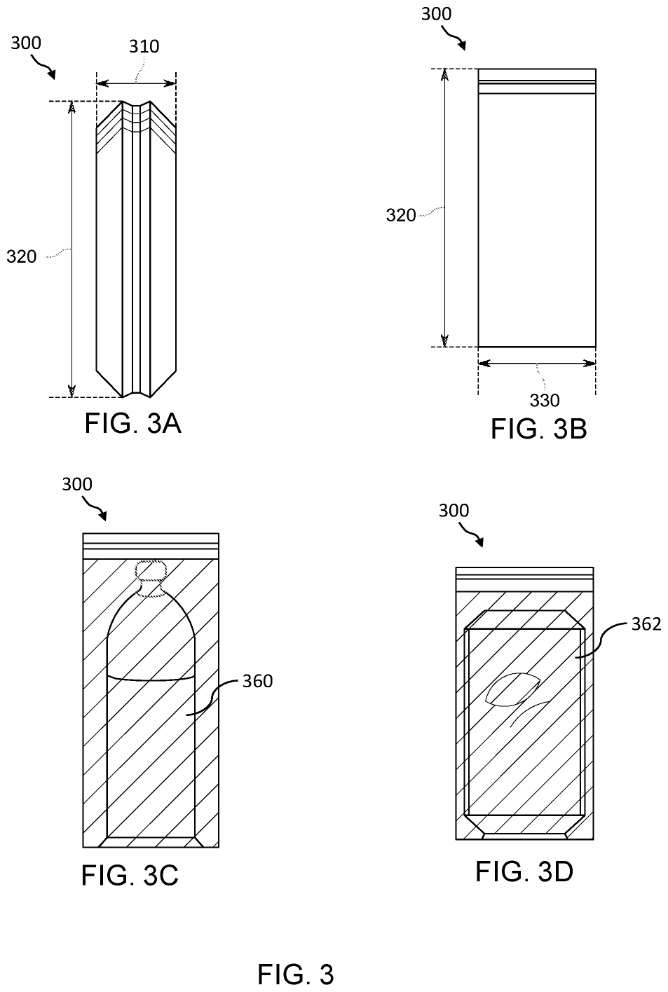

Referring now to FIG. 3, a bag 300 for sterilizing and disinfecting personal items in accordance with an embodiment of the present invention is shown, particularly for sterilizing and disinfecting bottled beverages or canned beverages.

FIG. 3A shows a side view of bag 300 having a width 310 and a height 320. Width 310 may vary according to need. Height 320 may also vary according to need such as the size of the bottled beverages or canned beverages.

Bag 300 may include two parts assembled in such a way that the bag is closed on three sides and open on one side and comprises a first layer, a second layer, and a third layer, wherein the first layer comprises an environmentally friendly degradable plastic linoleum; the second layer comprises flexible glass fiber insulation or any suitable insulator to provide insulation and support for the first layer; and the third layer comprises a disinfectant wet wipe wetted with one or more disinfectants approved by the World Health Organization, such as chlorine-based products, ethanol, or hydrogen peroxide.

FIG. 3B shows a front view of bag 300 having a length 330 and height 320. Length 330 may vary according to need. The side of bag 300 that is open may comprise an interlock design (not shown) for closing and opening the bag in a simplified way while maintaining a tight closure, thereby enabling effective sterilization and disinfection of bottled beverages or canned beverages inserted into bag 300.

FIG. 3C shows a front see-through view of bag 300, into which bottled beverage 360 has been inserted. A user need only move or rub the bag 300 with one or both hands until bottled beverage 360 is sterilized and disinfected easily and quickly.

FIG. 3D shows a front see-through view of bag 300, into which canned beverage 362 has been inserted. A user need only move or rub the bag 300 with one or both hands until canned beverage 360 is sterilized and disinfected easily and quickly.

In another embodiment, a bag for sterilizing and disinfecting personal items is provided that is divided into two parts: a first section that contains a wet wipe wetted with disinfecting materials such as alcohol, and a second section that contains a wet wipe wetted with water and materials extracted from herbs and/or fruits to rinse or purify personal items that have been treated in the first section. This rinsing step may also reduce the effects of alcohol-based disinfectants used in the first section on the personal items.

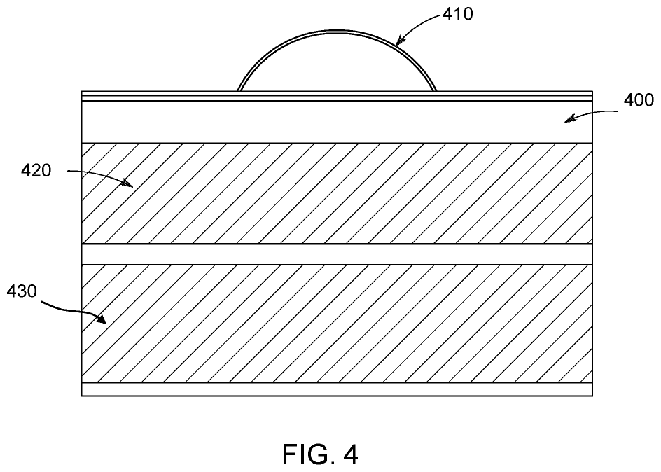

Referring now to FIG. 4, a bag 400 for sterilizing and disinfecting personal items in accordance with an embodiment of the present invention is shown. FIG. 4 shows a side view of bag 400 having a handle 410, a first pocket 420, and a second pocket 430. First pocket 420 comprises a first layer, a second layer, and a third layer, wherein the first layer comprises an environmentally friendly degradable plastic linoleum; the second layer comprises flexible glass fiber insulation or any suitable insulator to provide insulation and support for the first layer; and the third layer comprises a disinfectant wet wipe wetted with one or more disinfectants approved by the World Health Organization, such as chlorine-based products, ethanol, or hydrogen peroxide. Second pocket 430 comprises a first layer, a second layer, and a third layer, wherein the first layer comprises an environmentally friendly degradable plastic linoleum; the second layer comprises flexible glass fiber insulation or any suitable insulator to provide insulation and support for the first layer; and the third layer comprises a disinfectant wet wipe wetted with water and one or more extracts from fruits and/or herbs.

In another embodiment, a bag for sterilizing and disinfecting personal items is provided that is divided into multiple sections of varying size, wherein each section comprises a first layer, a second layer, and a third layer, wherein the first layer comprises an environmentally friendly degradable plastic linoleum; the second layer comprises flexible glass fiber insulation or any suitable insulator to provide insulation and support for the first layer; and the third layer comprises a disinfectant wet wipe wetted with one or more disinfectants approved by the World Health Organization, such as chlorine-based products, ethanol, or hydrogen peroxide. Alternatively, where light cleansing of the bottled beverages or canned beverages is desired, the disinfectant wet wipe is wetted with water and one or more extracts from fruits and/or herbs. In some aspects, some of the multiple sections comprise disinfectant wet wipes wetted with one or more disinfectants approved by the World Health Organization, such as chlorine-based products, ethanol, or hydrogen peroxide, while some of the multiple sections comprise disinfectant wet wipes wetted with water and one or more extracts from fruits and/or herbs.

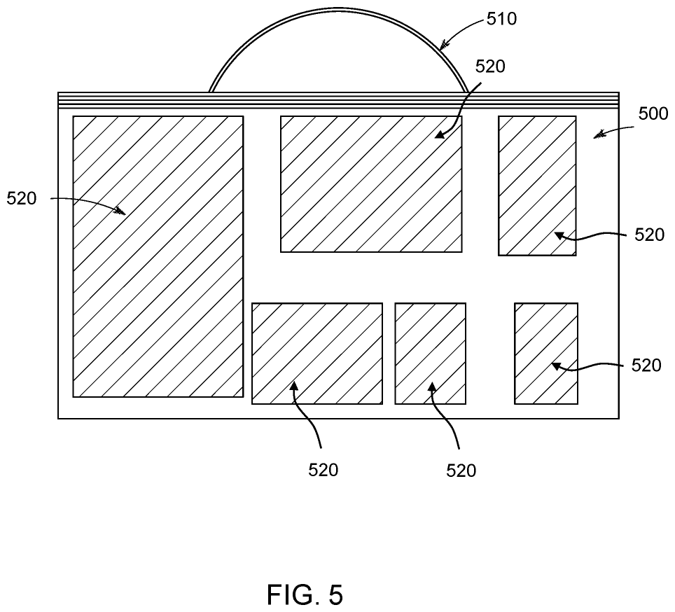

Referring now to FIG. 5, a bag 500 for sterilizing and disinfecting personal items in accordance with an embodiment of the present invention is shown. FIG. 5 shows a side view of bag 500 having a handle 510, and multiple pockets of varying size 520 Each of the multiple pockets of varying size 520 comprise a first layer, a second layer, and a third layer, wherein the first layer comprises an environmentally friendly degradable plastic linoleum; the second layer comprises flexible glass fiber insulation or any suitable insulator to provide insulation and support for the first layer; and the third layer comprises a disinfectant wet wipe wetted with one or more disinfectants approved by the World Health Organization, such as chlorine-based products, ethanol, or hydrogen peroxide.

In some embodiments, each of the multiple pockets of varying size 520 comprise a first layer, a second layer, and a third layer, wherein the first layer comprises an environmentally friendly degradable plastic linoleum; the second layer comprises flexible glass fiber insulation or any suitable insulator to provide insulation and support for the first layer; and the third layer comprises a disinfectant wet wipe wetted with water and one or more extracts from fruits and/or herbs.

In some embodiments, some of the multiple pockets of varying size 520 comprise a disinfectant wet wipe wetted with one or more disinfectants approved by the World Health Organization, such as chlorine-based products, ethanol, or hydrogen peroxide, and some of the multiple pockets of varying size 520 comprise a disinfectant wet wipe wetted with water and one or more extracts from fruits and/or herbs.

In one embodiment, a bag for sterilizing and disinfecting personal items is provided comprising:

a) a first layer comprising an environmentally friendly degradable plastic linoleum; and

b) a second layer comprising a disinfectant wet wipe wetted with:
i) one or more disinfectants approved by the World Health Organization; or
ii) water and one or more extracts from fruits and/or herbs; wherein the bag is configured to enable a user to rub one or more personal items placed inside the bag from outside the bag, thereby sterilizing and disinfecting the one or more personal items without the need for the user to touch the one or more personal items.

In another embodiment, the bag for sterilizing and disinfecting personal items comprises a layer comprising a disinfectant wet wipe reinforced with cotton or tree fibers and wetted with:
i) one or more disinfectants approved by the World Health Organization; or
ii) water and one or more extracts from fruits and/or herbs;
   wherein the bag is configured to enable a user to rub one or more personal items placed inside the bag from outside the bag, thereby sterilizing and disinfecting the one or more personal items without the need for the user to touch the one or more personal items.

In all embodiments of the bag for sterilizing and disinfecting personal items of the present invention, the bag and any pockets it contains may comprise locks.

In another embodiment, a method is provided for sterilizing and disinfecting a personal item, comprising: a) placing the personal item in a bag for sterilizing and disinfecting personal items as described herein; and b) rubbing the bag with one or both hands; thereby sterilizing and disinfecting the one or more personal items without the need for the user to touch the one or more personal items. In one aspect, the personal item is a mobile device and the bag is sized to receive the mobile device. In another aspect, the personal item comprises various articles and the bag is a shopping bag sized to receive the various articles. In another aspect, the personal item comprises a bottled beverage or a canned beverage and the bag is sized to receive the various articles the bottled beverage or the canned beverage.

In another embodiment, the method for sterilizing and disinfecting a personal item further comprises use of a bag for sterilizing and disinfecting personal items that is divided into two parts: a first section that contains a wet wipe wetted with disinfecting materials such as alcohol, and a second section that contains a wet wipe wetted with water and materials extracted from herbs and/or fruits to rinse or purify personal items that have been treated in the first section.

In another embodiment, the method for sterilizing and disinfecting a personal item further comprises use of a bag for sterilizing and disinfecting personal items that is divided into multiple sections of varying size, some of which comprise disinfectant wet wipes wetted with one or more disinfectants approved by the World Health Organization, such as chlorine-based products, ethanol, or hydrogen peroxide, and some of which comprise disinfectant wet wipes wetted with water and one or more extracts from fruits and/or herbs.

### Definitions

Following long-standing patent law convention, the terms “a,” “an,” and “the” refer to “one or more” when used in this application, including the claims. Thus, for example, reference to “a subject” includes a plurality of subjects, unless the context clearly is to the contrary (e.g., a plurality of subjects), and so forth.

Throughout this specification and the claims, the terms “comprise,” “comprises,” and “comprising” are used in a non-exclusive sense, except where the context requires otherwise. Likewise, the term “include” and its grammatical variants are intended to be non-limiting, such that recitation of items in a list is not to the exclusion of other like items that can be substituted or added to the listed items.

For the purposes of this specification and appended claims, unless otherwise indicated, all numbers expressing amounts, sizes, dimensions, proportions, shapes, formulations, parameters, percentages, quantities, characteristics, and other numerical values used in the specification and claims, are to be understood as being modified in all instances by the term “about” even though the term “about” may not expressly appear with the value, amount or range. Accordingly, unless indicated to the contrary, the numerical parameters set forth in the following specification and attached claims are not and need not be exact, but may be approximate and/or larger or smaller as desired, reflecting tolerances, conversion factors, rounding off, measurement error and the like, and other factors known to those of skill in the art depending on the desired properties sought to be obtained by the subject matter of the present invention. For example, the term “about,” when referring to a value can be meant to encompass variations of, in some embodiments ±100%, in some embodiments ±50%, in some embodiments ±20%, in some embodiments ±10%, in some embodiments ±5%, in some embodiments ±1%, in some embodiments+0.5%, and in some embodiments ±0.1% from the specified amount, as such variations are appropriate to perform the disclosed methods or employ the disclosed compositions.

Further, the term “about” when used in connection with one or more numbers or numerical ranges, should be understood to refer to all such numbers, including all numbers in a range and modifies that range by extending the boundaries above and below the numerical values set forth. The recitation of numerical ranges by endpoints includes all numbers, e.g., whole integers, including fractions thereof, subsumed within that range (for example, the recitation of 1 to 5 includes 1, 2, 3, 4, and 5, as well as fractions thereof, e.g., 1.5, 2.25, 3.75, 4.1, and the like) and any range within that range.

Although the foregoing subject matter has been described in some detail by way of illustration and example for purposes of clarity of understanding, it will be understood by those skilled in the art that certain changes and modifications can be practiced within the scope of the appended claims.

That which is claimed:

1. A bag for sterilizing and disinfecting personal items comprising:
a) a first layer comprising an environmentally friendly degradable plastic linoleum; and
b) a second layer comprising a disinfectant wet wipe wetted with:
i) one or more disinfectants approved by the World Health Organization; or
ii) water and one or more extracts from fruits and/or herbs;
wherein the bag is configured to enable a user to rub one or more personal items placed inside the bag from outside the bag, thereby sterilizing and disinfecting the one or more personal items without the need for the user to touch the one or more personal items.

2. The bag of claim 1, wherein the one or more disinfectants approved by the World Health Organization comprise chlorine-based products, ethanol, and/or hydrogen peroxide.

3. The bag of claim 1, wherein the wet wipe comprises cotton or tree fibers.

4. The bag of claim 1; wherein the personal items comprise a mobile device, and wherein the bag is sized to accommodate a desired make and model of mobile device.

5. The bag of claim 1, wherein the bag comprises a lock.

6. A method for sterilizing and disinfecting a personal item, comprising placing the personal item in the bag of claim 1, and nabbing the bag with one or both hands; thereby sterilizing and disinfecting the personal item without the need for the user to touch the one or more personal items.

7. The method of claim 6, wherein the personal item is a mobile device and the bag is sized to receive the mobile device.

8. A bag for sterilizing and disinfecting personal items comprising a layer comprising a disinfectant wet wipe wetted with:
i) one or more disinfectants approved by the World Health Organization; or
ii) water and one or more extracts from fruits and/or herbs;
wherein the bag is configured to enable a user to rub one or more personal items placed inside the bag from outside the bag, thereby sterilizing and disinfecting the one or more personal items without the need for the user to touch the one or more personal items.

9. The bag of claim 8, wherein the one or more disinfectants approved by the World Health Organization comprise chlorine-based products, ethanol, and/or hydrogen peroxide.

10. The bag of claim 8, wherein the wet wipe comprises cotton or tree fibers.

11. The bag of claim 8, wherein the personal items comprise a mobile device, and wherein the bag is sized to accommodate a desired make and model of mobile device.

12. The bag of claim 8, wherein the bag comprises a lock.

13. A method for sterilizing and disinfecting a personal item, comprising placing the personal item in the bag of claim 8, and rubbing the bag with one or both hands; thereby sterilizing and disinfecting the personal item without the need for the user to touch the one or more personal items.

14. The method of claim 13, wherein the personal item is a mobile device and the bag is sized to receive the mobile device.
